(19) United States
(12) Patent Application Publication
(10) Pub. No.: US 2024/0131207 A1
(43) Pub. Date: Apr. 25, 2024

(54) NUCLIDE-LABELED INHIBITORY PEPTIDE, AND PREPARATION METHOD THEREFOR AND USE THEREOF

(71) Applicant: SUZHOU UNIVERSITY, Suzhou (CN)

(72) Inventors: Kai YANG, Suzhou (CN); Xiumin SHI, Suzhou (CN)

(73) Assignee: SUZHOU UNIVERSITY, Suzhou (CN)

(21) Appl. No.: 18/510,683

(22) Filed: Nov. 16, 2023

Related U.S. Application Data
(63) Continuation of application No. PCT/CN2023/081067, filed on Mar. 13, 2023.

(30) Foreign Application Priority Data
May 26, 2022 (CN) 202210578560.X

(51) Int. Cl. A61K 51/08; A61P 35/00; C07B 59/00; C07K 1/20; C07K 14/47

(52) U.S. Cl. CPC A61K 51/088; A61P 35/00; C07B 59/008; C07K 1/20; C07K 14/4703; A61K 2121/00; A61K 2123/00; C07B 2200/05

(57) ABSTRACT

A nuclide-labeled inhibitory peptide, and a preparation method therefor and use thereof are provided. The nuclide-labeled inhibitory peptide is prepared by labeling ASF1a peptide with ⁶⁸Ga/¹⁷⁷Lu by DOTA, and an amino acid sequence of the ASF1a peptide is YGRKKRRQRRRCASTEEKWARLARRIAGAGGVTLDGFGGCA (as shown in SEQ ID NO: 1). The ⁶⁸Ga labeled ASF1a inhibitory peptide of the present invention displays the expression level of ASF1a of a tumor through PET/CT imaging, has good imaging sensitivity, can specifically screen high-expression and low-expression individuals, and achieves noninvasive prediction of the efficacy of tumor immunotherapy. The ¹⁷⁷Lu-labeled ASF1a inhibitory peptide provides a novel and effective therapeutic strategy for a tumor that highly expresses ASF1a and is not effective for immunotherapy.

### CROSS REFERENCE TO THE RELATED APPLICATIONS

This application is a continuation application of International Application No. PCT/CN2023/081067, filed on Mar. 13, 2023, which is based upon and claims priority to Chinese Patent Application No. 202210578560.X, filed on May 26, 2022, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBRZBC150_Sequence_Listing.xml, created on Nov. 1, 2023, and is 2,173 bytes in size.

### TECHNICAL FIELD

The present invention relates to the technical field of tumor prognosis, and in particular to a nuclide-labeled inhibitory peptide, and a preparation method therefor and use thereof.

### BACKGROUND

In recent years, immunotherapy has revolutionized the field of cancer research by using an immune system to fight cancer. Immunotherapy has become an important approach for cancer treatment today. However, in fact only a small number (less than 20%) of patients can benefit from anti-PD-1/PD-L1 immunotherapy, and serious adverse events occurred in 20-40% of these patients. It is important to identify which patients are more likely to benefit from immune checkpoint blockade (ICB) to maximize efficacy and minimize toxicity.

ASF1a promotes suppression of tumor immunity. ASF1a is overexpressed in a variety of primary human tumors including melanoma and LUAD. It has been shown that high expression of ASF1a is associated with a significantly poorer prognosis in patients with hepatocellular carcinoma. ASF1a is a potential therapeutic target.

ASF1 is a histone H3-H4 chaperone conserved from yeast to human cells. ASF1a and ASF1b are mammalian isoforms involved in DNA replication-coupled and DNA replication-uncoupled nucleosome assembly pathways. ASF1 also plays a role in gene transcription regulation. For example, ASF1a resolves bivalent chromatin domains during embryonic stem cell differentiation to induce lineage specific genes. Functional and mechanistic studies indicate that ASF1a deficiency sensitizes LUAD tumors to anti-PD-1 therapy by promoting M1-like macrophage polarization and enhancing T cell activation. ASF1a is a negative regulator of immunotherapy, the expression level of ASF1a of a tumor is dynamically monitored through the visualization of a designed PET probe, and the treatment strategy of a patient with a cancer is formulated according to the expression level of ASF1a.

Therefore, how to design and provide a nuclide-labeled polypeptide targeting ASF1a for an ASF1a target and apply the nuclide-labeled polypeptide to prognosis of tumor immunotherapy is an urgent problem to be solved by those skilled in the art.

### SUMMARY

An objective of the present invention is to design and provide use of a gallium-labeled polypeptide targeting ASF1a in a PET/CT imaging agent for predicting tumor immunotherapy drug resistance for an ASF1a target. The present invention can clearly develop and predict the efficacy of immunotherapy by 1.11-3.7 MBq of administration, can repeatedly develop in a short period and dynamically monitor immunotherapy, and simultaneously provides an effective treatment strategy by performing isotope-labeled polypeptide targeted therapy on screened immune-resistant individuals with high expression of ASF1a. The method of the present invention is not limited to skin melanoma, but is more suitable for tumors with high expression of ASF1a, such as lung cancer, lung metastatic cancer, and breast cancer.

In order to achieve the above objective, the present invention provides the following technical solutions:

The present invention provides a nuclide-labeled inhibitory peptide prepared by labeling ASF1a peptide with ⁶⁸Ga or ¹⁷⁷Lu by 1,4,7,10-tetraazacyclododecane-1,4,7,10-tetraacetic acid (DOTA), wherein the ASF1a peptide has an amino acid sequence of YGRKKRRQRRRCASTEEKWARLARRIAGAGGVTLDGFGGCA (as shown in SEQ ID NO: 1) (ASF1a Peptide, AP1) and a molecular weight (MW) of 4952.62. The nuclide-labeled position is a position represented by [symbol] in the schematic diagram 1 on the tetraazacyclo ring of DOTA.

The present invention further provides a preparation method for the nuclide-labeled inhibitory peptide, comprising the following steps:
(1) mixing the DOTA-coupled ASF1a peptide with a nuclide solution to obtain a mixed solution, mixing the mixed solution with sodium acetate, adjusting pH, and placing in a water bath to obtain a reaction solution;
(2) enabling the reaction solution obtained in the step (1) to pass through a chromatography column, and collecting a product.

Preferably, the nuclide solution in the step (1) is a ⁶⁸GaCl₃ solution or a ¹⁷⁷LuCl₃/HCl solution, and a radiation amount of the ⁶⁸GaCl₃ solution or the ¹⁷⁷LuCl₃/HCl solution is independently 111-185 MBq.

Preferably, a preparation method for the ⁶⁸GaCl₃ solution comprises: rinsing a ⁶⁸Ge—⁶⁸Ga generator by hydrochloric acid, and collecting an intermediate product ⁶⁸GaCl₃; wherein an amount of the hydrochloric acid is 4 mL, and the intermediate product is ⁶⁸GaCl₃ that is 2^{nd} to 3^{rd} mL flowing out of the ⁶⁸Ge—⁶⁸Ga generator.

Preferably, the hydrochloric acid has a concentration of 0.04-0.06 M, and the ⁶⁸Ge—⁶⁸Ga generator has a flow rate of 0.8-1.2 mL/min.

Preferably, the DOTA-coupled ASF1a peptide in the step (1) has a concentration of 0.8-1.2 mg/mL, and a volume ratio of the DOTA-coupled ASF1a peptide to the nuclide solution is (0.01-0.03):(1.00-3.00).

Preferably, a volume ratio of the DOTA-coupled ASF1a peptide to the sodium acetate in the step (1) is (15-25):(250-350), the sodium acetate has a concentration of 0.23-0.27 M, and the pH is adjusted to 3.8-4.2.

Preferably, the water bath in step (1) has a temperature of 93-97° C., and the chromatography column in the step (2) is a C18 small column.

Preferably, when the nuclide solution is a ⁶⁸GaCl₃ solution, the water bath is performed for 8-12 min, and when the nuclide solution is a ¹⁷⁷LuCl₃/HCl solution, the water bath is performed for 25-35 min.

The present invention further provides use of the nuclide-labeled inhibitory peptide or a nuclide-labeled inhibitory peptide prepared by the preparation method for a nuclide-labeled inhibitory peptide in preparing a PET/CT imaging agent, wherein the tumor is one of melanoma, lung cancer, lung metastatic cancer, and breast cancer.

Compared with the prior art, the present invention has the following beneficial effects:
1. The ⁶⁸Ga is produced by a ⁶⁸Ge—⁶⁸Ga generator, can be repeatedly produced every 4 hours, can be used for more than 1 year, has low nuclide cost, has a half-life of only 68 minutes and low radiation dose, meets the requirement of short-term repeated dynamic monitoring, and can be used as a dynamic monitoring imaging agent of tumor immunotherapy.
2. The present invention establishes a method for labeling ASF1a inhibitory peptide (AP1) with ⁶⁸Ga/¹⁷⁷Lu by using ⁶⁸GaCl₃ produced by a ⁶⁸Ge—⁶⁸Ga generator or purchased ¹⁷⁷LuCl₃ and designed ASF1a inhibitory peptide, evaluates the pharmacological characteristics and the biological characteristics of the ASF1a inhibitory peptide in a B16F10 tumor model mouse, is further used in the ASF1a targeted imaging study, and analyzes the correlation of the imaging result with immunotherapy. The efficacy of radionuclide-targeted therapy on screened ASF1a individuals is evaluated. The preclinical study shows that the labeling rate of ⁶⁸Ga-AP1 is 81.98±7.55%, the labeling rate of ¹⁷⁷Lu-AP1 is 78.34±13.59%, the radiochemical purity of the product measured by an HPLC method is greater than 95%, and the product has good stability within 24 hours.
3. The designed and synthesized ASF1a peptide has good biocompatibility, when the maximum concentration of the polypeptide is 100 μg/mL, the survival rate of cells in 24 hours is 94.73±10.96%, and the survival rate of cells in 48 hours is 102.73±5.76%, and no significant difference exists among the concentrations. The synthesized ⁶⁸Ga-AP1 is used as a PET/CT imaging probe, the cell survival rate is 95.31±9.05% when the radioactivity is at most 200 μCi/mL, and no significant difference exists in dose groups. The results show that the polypeptide and the PET/CT imaging probe have good biocompatibility.
4. It is studied that the uptake of the synthesized ⁶⁸Ga-AP1 and ¹⁷⁷Lu-AP1 in B16F10 cells can be inhibited by AP1, and the difference is statistically significant, which indicates that the ⁶⁸Ga-AP1 and ¹⁷⁷Lu-AP1 can be specifically uptaken at the cellular level.
5. It is studied that the synthesized ¹⁷⁷Lu-AP1 has a more excellent killing effect in B16F10 cells than the free ¹⁷⁷LuCl₃, the difference is statistically significant, and the inhibition effect on the tumor cell proliferation can be continuously observed in the following 24 hours and 48 hours. In the experiment, after two groups of medicaments are added into B16F10 cells and the cells are incubated for 24 hours, a normal culture medium is used, and ¹⁷⁷Lu-AP1 is significantly superior to ¹⁷⁷LuCl₃ when the dosage is 100 μCi/mL, and the inhibition effect is more significant along with the increase of the dosage. When the radioactive dose is 600 μCi/mL, after 24 hours, the cell survival rate of the ¹⁷⁷Lu-AP1 group is 65.31±13.64%, and the cell survival rate of the ¹⁷⁷LuCl₃ group is 82.19±16.69%; after 48 hours, the cell survival rate of the ¹⁷⁷Lu-AP1 group is 64.59±8.28%, and the cell survival rate of the ¹⁷⁷LuCl₃ group is 86.98.19±3.22%.
6. In a B16F10 tumor-bearing mouse model, ⁶⁸Ga-AP1 imaging shows that different individuals have different levels of uptake, the optimal imaging time is 3.5-5.5 hours after the injection of the imaging agent, and the in vivo biodistribution data also shows that the imaging agent is mainly metabolized through liver and kidney, and after 5.5 hours, the tumor % ID/g in a tumor high-intake group is 18.95±0.2479%, the tumor % ID/g in a tumor low-intake group is 5.243±1.734%, and the difference is statistically significant. In the hemodynamic analysis, the half-discharge time of ⁶⁸Ga-AP1 in the blood is 2.1933 hours and the mean residence time (MRT) in vivo is 3.1643 hours.
7. The B16F10 tumor model is analyzed for correlation of the ratio of maximum tumor uptake to maximum contralateral muscle uptake in early imaging with the efficacy of the immunosuppressant BMS-1 in mice. All mice in the treatment group are intraperitoneally injected with BMS-1 starting on day 4 after tumor bearing, with 0.05 mg/mouse every 3 days, and tumor volumes are recorded; the mice are observed to the end of day 16, and subjected to ⁶⁸Ga-AP1 imaging during the course of treatment on day 7 or day 10, respectively, and the ratio T/N of tumor uptake/maximum uptake in contralateral muscle of the mice is recorded. The immunotherapy is considered effective if the tumor volume is less than 1000 mm³ on day 16. The T/N ratio of this group is 1.11±0.2362, and the T/N ratio of the immunotherapy-ineffective group is 2.32±0.5997, and the difference between the two groups is statistically significant. The study shows that the higher the uptake is in ⁶⁸Ga-AP1 imaging, the more likely the immunotherapy is ineffective, and the method is expected to be used for non-invasively predicting the individuals with ineffective immunotherapy or dynamically monitoring during the immunotherapy process to guide the therapy.
8. The individuals with the T/N ratio of ⁶⁸Ga-AP1 imaging being greater than 2.32 are screened for BMS-1 alone and combined with ¹⁷⁷Lu-AP1 for treatment, ¹⁷⁷Lu-AP1 has significant inhibition effect on the proliferation of tumors, the tumor inhibition rate is 63.94%, and the difference is statistically significant.

In conclusion, the ⁶⁸Ga labeled ASF1a inhibitory peptide of the present invention displays the expression level of ASF1a of a tumor through PET/CT imaging, has good imaging sensitivity, can specifically screen high-expression and low-expression individuals, and achieves noninvasive prediction of the efficacy of tumor immunotherapy. The ¹⁷⁷Lu-labeled ASF1a inhibitory peptide provides a novel and effective therapeutic strategy for a tumor that highly expresses ASF1a and is not effective for immunotherapy.

### BRIEF DESCRIPTION OF DRAWINGS

In order to more clearly illustrate the technical solutions in the embodiments of the present invention or in the prior art, the drawings required to be used in the description of the embodiments or the prior art are briefly introduced below. It is obvious that the drawings in the description below are merely embodiments of the present invention, and those of ordinary skill in the art can obtain other drawings according to the drawings provided without creative efforts.

FIG. 1 is a schematic diagram of a ⁶⁸Ga/¹⁷⁷Lu-labeled AP1 polypeptide;

FIG. 2 is a diagram of identification and purification of a ⁶⁸Ga/¹⁷⁷Lu-labeled AP1 product;

FIGS. 3A-3B show biological safety of AP1 at 24 hours and 48 hours after incubation in B16F10 cells for 24 hours, where FIG. 3A shows the inhibition of cell growth at 24 hours after incubation of AP1 in B16F10 cells for 24 hours, and FIG. 3B shows the inhibition rate of cell growth at 48 hours after incubation of AP1 in B16F10 cells for 24 hours;

FIG. 4 shows biological safety of ⁶⁸Ga-AP1 at different doses;

FIGS. 5A-5B show a competitive binding and inhibition experiment between ⁶⁸GaAP1 and AP1;

FIG. 6 shows a specific uptake and inhibition of ¹⁷⁷Lu-AP1 in B16F10 cells;

FIGS. 7A-7B show inhibitory effect of ¹⁷⁷Lu-AP1 on B16F10 cell growth, where FIG. 7A shows cell growth inhibition rate at 24 hours after incubation for 24 hours, and FIG. 7B shows cell growth inhibition rate at 48 hours after incubation for 24 hours;

FIGS. 8A-8C show in vivo targeting and specificity of a ⁶⁸Ga-AP1 imaging probe, where FIG. 8A shows PET/CT imaging of an individual that is sensitive to immunotherapy; FIG. 8B shows PET/CT imaging of an individual that is not insensitive to immunotherapy, and FIG. 8C shows PET/CT imaging of a high-uptake tumor that is insensitive to immunotherapy after addition of AP1 inhibition;

FIGS. 9A-9C show in vivo biodistribution and hemodynamic characteristics of FIG. 9A ⁶⁸Ga-AP1 imaging probe, where a shows biodistribution of ⁶⁸Ga-AP1 in major organs in a body, FIG. 9B shows distributions of ⁶⁸Ga-AP1 in ASF1a high-expression and low-expression tumors, respectively, and FIG. 9C shows pharmacokinetics of ⁶⁸Ga-AP1 in vivo;

FIGS. 10A-10B show the correlation between different expressions of ASF1a and the efficacy of immunotherapy in a melanoma B16F10 model, where FIG. 10A shows a tumor volume growth curve of mice after treatment with the immunosuppressant BMS-1, purple represents an effective immunosuppressive treatment group, and black represents an ineffective immunosuppressive treatment group; FIG. 10B shows ratios of the tumors to contralateral muscle uptake in different response groups of immunotherapy;

FIG. 11 shows tumor volume growth curves of immunotherapy-insensitive individuals treated with BMS-1 alone and BMS-1 combined with ¹⁷⁷Lu-AP1.

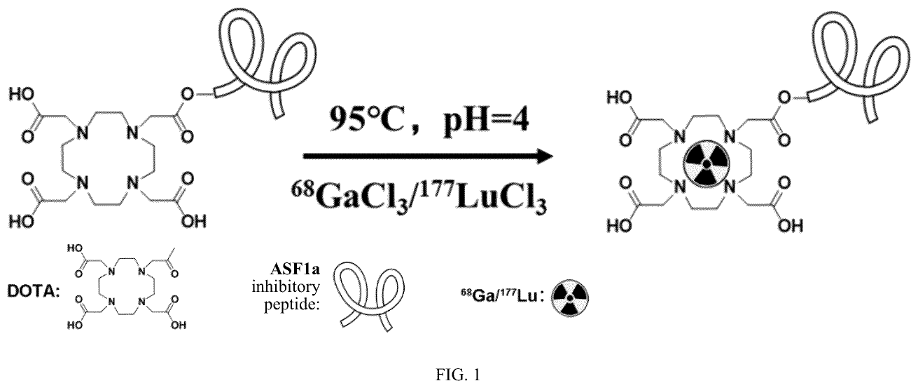

### DETAILED DESCRIPTION OF EMBODIMENTS

The present invention provides a nuclide-labeled inhibitory peptide prepared by labeling ASF1a peptide with ⁶⁸Ga or ¹⁷⁷Lu by DOTA, wherein the ASF1a peptide has an amino acid sequence of YGRKKRRQRRRCASTEEKWARLARRIAGAGGVTLDGFGGCA (as shown in SEQ ID NO: 1) (ASF1a Peptide, AP1) and a molecular weight (MW) of 4952.62. The nuclide-labeled position is a position represented by [symbol] in the schematic diagram 1 on the tetraazacyclo ring of DOTA.

The present invention further provides a preparation method for the nuclide-labeled inhibitory peptide, comprising the following steps:
(1) mixing the DOTA-coupled ASF1a peptide with a nuclide solution to obtain a mixed solution, mixing the mixed solution with sodium acetate, adjusting pH, and placing in a water bath to obtain a reaction solution;
(2) enabling the reaction solution obtained in the step (1) to pass through a chromatography column, and collecting a product.

In the present invention, the nuclide solution in the step (1) is a ⁶⁸GaCl₃ solution or a ¹⁷⁷LuCl₃/HCl solution, preferably a ⁶⁸GaCl₃ solution.

In the present invention, a radiation amount of the ⁶⁸GaCl₃ solution or the ¹⁷⁷LuCl₃/HCl solution in the step (1) is independently 111-185 MBq, preferably 121-175 MBq, further preferably 131-165 MBq, and more preferably 145 MBq.

In the present invention, a preparation method for the ⁶⁸GaCl₃ solution comprises: rinsing a ⁶⁸Ge—⁶⁸Ga generator by hydrochloric acid, and collecting an intermediate product ⁶⁸GaCl₃; wherein an amount of the hydrochloric acid is 4 mL, and the intermediate product is ⁶⁸GaCl₃ that is 2^{nd} to 3^{rd} mL flowing out of the ⁶⁸Ge—⁶⁸Ga generator.

In the present invention, the hydrochloric acid has a concentration of 0.04-0.06 M, preferably 0.05 M.

In the present invention, the flow rate of the ⁶⁸Ge—⁶⁸Ga generator is 0.8-1.2 mL/min, preferably 0.9-1.1 mL/min, and further preferably 1 mL/min.

In the present invention, the DOTA-coupled ASF1a peptide in the step (1) has a concentration of 0.8-1.2 mg/mL, preferably 0.9-1.1 mg/mL, and further preferably 1 mg/mL.

In the present invention, a volume ratio of the DOTA-coupled ASF1a peptide to the nuclide solution in the step (1) is (0.01-0.03):(1.00-3.00), preferably 0.02:(1.0-2.5), and further preferably 0.02:2.05.

In the present invention, a volume ratio of the DOTA-coupled ASF1a peptide to the sodium acetate in the step (1) is (15-25):(250-350), preferably (17-23):(270-330), further preferably (19-21):(290-310), and more preferably 20:300.

In the present invention, the sodium acetate in the step (1) has a concentration of 0.23-0.27 M, preferably 0.24-0.26 M, and further preferably 0.25 M.

In the present invention, the pH in the step (1) is adjusted to 3.8-4.2, preferably 3.9-4.1, and further preferably 4.0.

In the present invention, the water bath in step (1) has a temperature of 93-97° C., preferably 94-96° C., and further preferably 95° C.

In the present invention, the chromatography column in the step (1) is a C18 small column.

In the present invention, when the nuclide solution is a ⁶⁸GaCl₃ solution, the water bath is performed for 8-12 min, preferably 9-11 min, and further preferably 10 min.

In the present invention, when the nuclide solution is a ¹⁷⁷LuCl₃/HCl solution, the water bath is performed for 25-35 min, preferably 27-33 min, further preferably 29-31 min, and more preferably 30 min.

The present invention further provides use of the nuclide-labeled inhibitory peptide or a nuclide-labeled inhibitory peptide prepared by the preparation method for a nuclide-labeled inhibitory peptide in preparing a PET/CT imaging agent, wherein the tumor is one of melanoma, lung cancer, lung metastatic cancer, and breast cancer, preferably melanoma.

The technical solutions provided by the present invention will be described in detail below with reference to the examples, which, however, should not be construed as limiting the scope of the present invention.

### Main Instruments or Equipment

| | |
|---|---|
| PET/CT | SIEMENS, Germany |
| ⁶⁸Ge—⁶⁸Ga generator | ITG, Germany |
| Multifunctional microplate reader | Bio-Tek, USA |
| 2480 WIZARD2 Automatic Gamma Counter | PerkinElmer, USA |
| High performance liquid chromatograph | Shimadzu, Japan |
| LC-20AT | |

### Reagents and Consumables

| | |
|---|---|
| BMS-1 | MCE, China |
| CCK8 kit | Beyotime, China |
| C18 column | Sep-Pak, Ireland |

0.05M HCl, 528 μL of 30% HCl was diluted to a volume of 100 mL by
adding ultrapure water
0.25M sodium acetate (NaOAc), 2.05 g of anhydrous sodium acetate
powder was weighed and dissolved in 100 mL of ultrapure water, and
dissolved by ultrasonic

### EXAMPLE 1

### Synthesis of ASF1a Peptide, AP1

(1) Estimating a feeding amount of each amino acid according to the weight and molecular weight of the target polypeptide, wherein each amino acid and DOTA use protected raw materials.
(2) Placing 2-Cl(Trt)-Cl resin into a 150 mL reactor, and adding 80 mL of DCM for soaking for 2 h.
(3) Washing the resin with DMF and then draining, and repeating 4 times to drain the resin.
(4) Weighing Fmoc-Ala-OH (the first amino acid at the C terminal, CAS No. 154445-77-9)+80 mL of DCM and DIEA and adding into a reactor, and then placing the reactor into a shaker at 30° C. for reaction for 2 h.
(5) Adding 0.5 mL of DIEA and 0.5 mL of methanol through a pipette for reaction for 20 min, and blocking unreacted groups on the resin.
(6) Adding an appropriate amount of piperidine solution (piperidine/DMF=1:4) in a volume ratio of 20% which is 3 times of the volume of the resin into a reactor, reacting for 20 min, removing an Fmoc protecting group, washing with DMF 4 times after the protection is removed, and then draining;
(7) Taking 10-20 resins in the reactor by using a long-neck pipette, detecting by using a ninhydrin method, wherein if the resins are colored, indicating that deprotection is successful; if no color is developed, repeating the deprotection-washing-detection operation.
(8) Weighing Fmoc-Cys(trt)-OH (the second amino acid at the C-terminal, with a molar amount that is 3 times that of the first amino acid) and an appropriate amount of HOBT and DIC and adding to a reactor, and then placing the reactor into a shaker at 30° C. for reaction for 1 h.
(9) Washing the resin with DMF and then draining, and repeating 4 times to drain the resin.
(10) Taking 10-20 resins in the reactor by using a long-neck pipette, detecting by using a ninhydrin method, wherein if the resins are colored, indicating that condensation is incomplete, continuing to react; if the resins are colorless, indicating that the reaction is complete.
(11) Adding a piperidine solution (piperidine/DMF=1:4) in a volume ratio of 20% which is 3 times of the volume of the resin into a reactor, reacting for 20 min, removing an Fmoc protecting group, washing with DMF 4 times after the protection is removed, and then draining; taking 10-20 resins in the reactor by using a long-neck pipette, detecting by using a ninhydrin method, wherein if the resins are colored, indicating that deprotection is successful; if no color is developed, repeating the deprotection-washing-detection operation.
(12) Linking the remaining amino acids and DOTA in sequence according to the steps 8-11.
(13) Cutting off all the polypeptide protecting groups by using a cutting reagent, cutting off the polypeptide protecting groups from the resin, adding a cutting fluid containing the polypeptide into the glacial ethyl ether, and normally settling the polypeptide in the glacial ethyl ether in a precipitation state; after the polypeptide is settled, centrifuging the system in a low-temperature centrifuge to remove a supernatant; resuspending and washing the precipitate with glacial ethyl ether, centrifuging again to remove the supernatant, and washing away residual impurities; repeating the operations 4 times to obtain a crude product of the target polypeptide.
(14) Separating the target peptide fragment from impurities by a high performance liquid chromatograph (HPLC), freeze-drying the inoculated target peptide fragment solution into powder to obtain the DOTA-coupled ASF1a peptide (ASF1a Peptide, AP1) with a molecular weight (MW) of 4952.62, and sending the peptide to QC quality inspection.

### EXAMPLE 2

A nuclide-labeled inhibitory peptide is prepared by labeling ASF1a peptide with ⁶⁸Ga by DOTA, wherein an amino acid sequence of the ASF1a peptide is YGRKKRRQRRRCASTEEKWARLARRIAGAGGVTLDGFGGCA (as shown in SEQ ID NO: 1).

A preparation method for the nuclide-labeled inhibitory peptide comprises the following steps:
(1) rinsing a ⁶⁸Ge—⁶⁸Ga generator with 4 mL of hydrochloric acid at a concentration of 0.05 M (with a flow rate of 0.8 mL/min), and collecting an intermediate product ⁶⁸GaCl₃ of the 2^{nd} to 3^{rd} mL;
(2) mixing 15 μL of DOTA-coupled ASF1a peptide (with a concentration of 1 mg/mL) with 1 mL of a ⁶⁸GaCl₃ solution (with a radiation amount of 111 MBq) to obtain a mixed solution, mixing the mixed solution with 250 μL of sodium acetate (with a concentration of 0.23 M), adjusting the pH to 3.8, and placing in a metal bath at 93° C. for 8 min to obtain a reaction solution;
(3) dropwise activating the C18 small column by using 5 mL of 70% ethanol, washing by using 5 mL of normal saline, then pushing 10 mL of air, adding the reaction solution obtained in the step (2), washing by using 1 mL of normal saline to remove residual water (collected as free radionuclide), washing the C18 small column by using 0.3 mL of 60% ethanol, and collecting a product, namely the purified labeled product.

### EXAMPLE 3

A nuclide-labeled inhibitory peptide is prepared by labeling ASF1a peptide with ¹⁷⁷Lu by DOTA, wherein an amino acid sequence of the ASF1a peptide is YGRKKRRQRRRCASTEEKWARLARRIAGAGGVTLDGFGGCA (as shown in SEQ ID NO: 1).

A preparation method for the nuclide-labeled inhibitory peptide comprises the following steps:
(1) mixing 25 μL of DOTA-coupled ASF1a peptide (with a concentration of 1.2 mg/mL) with 1.1 mL of a ¹⁷⁷LuCl₃/HCl solution (with a radiation amount of 185 MBq) to obtain a mixed solution, mixing the mixed solution with 350 μL of sodium acetate (with a concentration of 0.27 M), adjusting the pH to 4.2, and placing in a metal bath at 97° C. for 35 min to obtain a reaction solution;
(2) dropwise activating the C18 small column by using 6 mL of 70% ethanol, washing by using 6 mL of normal saline, adding the reaction solution obtained in the step (1), washing by using 3 mL of normal saline to remove residual water (collected as free radionuclide), washing the C18 small column by using 0.4 mL of 60% ethanol, and collecting a product, namely the purified labeled product.

### EXAMPLE 4

A nuclide-labeled inhibitory peptide is prepared by labeling ASF1a peptide with ⁶⁸Ga by DOTA, wherein an amino acid sequence of the ASF1a peptide is YGRKKRRQRRRCASTEEKWARLARRIAGAGGVTLDGFGGCA (as shown in SEQ ID NO: 1).

A preparation method for the nuclide-labeled inhibitory peptide comprises the following steps:
(1) rinsing a ⁶⁸Ge—⁶⁸Ga generator with 4 mL of hydrochloric acid at a concentration of 0.05 M (with a flow rate of 1 mL/min), and collecting an intermediate product ⁶⁸GaCl₃ of the 2^{nd} to 3^{rd} mL;
(2) mixing 20 μL of DOTA-coupled ASF1a peptide (with a concentration of 1 mg/mL) with 2.05 mL of a ⁶⁸GaCl₃ solution (with a radiation amount of 145 MBq) to obtain a mixed solution, mixing the mixed solution with 500 μL of sodium acetate (with a concentration of 0.25 M), adjusting the pH to 4, placing in a metal bath at 95° C. for 10 min to obtain a reaction solution, and cooling to room temperature;
(3) dropwise activating the C18 small column by using 5 mL of 70% ethanol, washing by using 5 mL of normal saline, then pushing 13 mL of air, adding the reaction solution obtained in the step (2), washing by using 2 mL of normal saline to remove residual water (collected as free radionuclide), washing the C18 small column by using 0.35 mL of 60% ethanol, and collecting a product, namely the purified labeled product.

### EXPERIMENTAL EXAMPLE 1

### Identification and Stability Analysis of ⁶⁸Ga/¹⁷⁷Lu Labeled Product

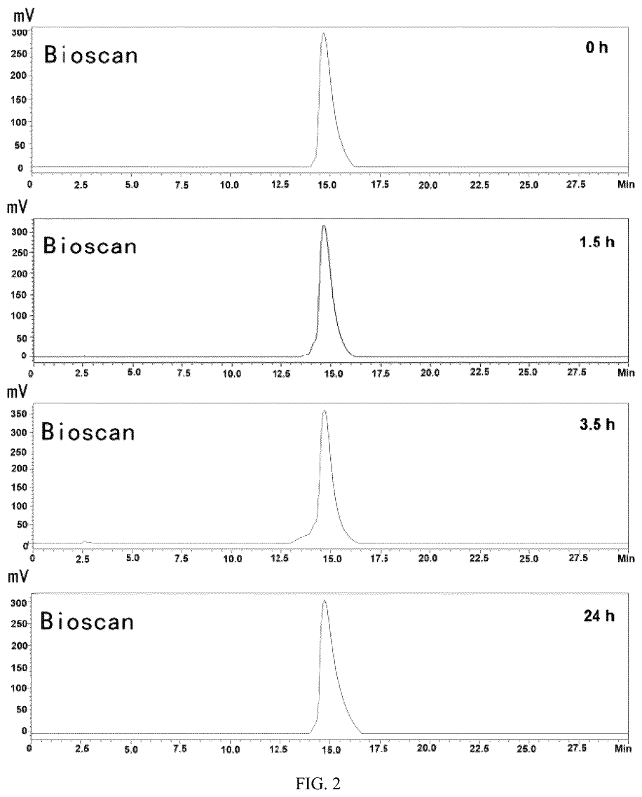

(1) The HPLC mobile phases were water and acetonitrile (each containing 0.1% TFA), and the gradient was set from 20% to 50% acetonitrile concentration over a period of 30 minutes.
(2) The injection analysis of ⁶⁸Ga/¹⁷⁷Lu-AP1 in PBS at different time points (0, 1.5, 3.5, and 24 h) under the same condition is shown in FIG. 2 (identification and purification of ⁶⁸Ga/¹⁷⁷Lu-labeled AP1 product) (The radiation peak of the product is mainly the peak of the polypeptide. Since the half-life of ⁶⁸Ga is only 68 minutes, and the amount of radiation injected is very small, the stability data is determined by ¹⁷⁷Lu (6.71 days) with a longer half-life).

### AP1 Safety Analysis

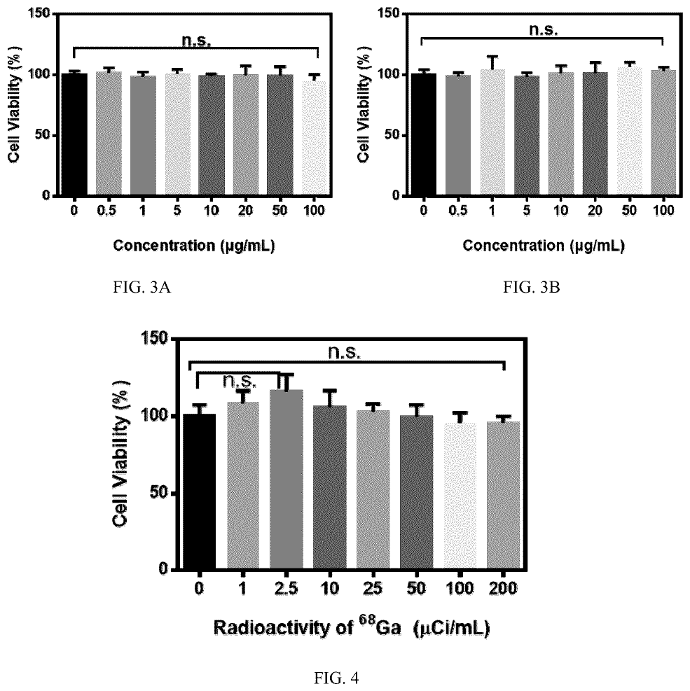

(1) The skin melanoma B16F10 cells of mice in the logarithmic growth phase were taken and digested by using 0.25% pancreatin, and the cell concentration was adjusted to 6×10⁴/mL; the cells were plated in a 96-well plate at a volume of 100 μL per well, the cell concentration was 6×10³ cells per well, and the cells were incubated overnight in an incubator at 37° C./5% CO₂;
(2) the medium was replaced for each group of cells, and 100 μL of a culture solution with a specified concentration was added, wherein the concentration of each dose was 0, 0.5, 1, 5, 10, 20, 50 and 100 μg/mL; after 24 h, a DMEM medium (containing 10% of North American fetal bovine serum and 1% of double antibody) was used, and the culture was continued;
(3) 10 μL of CCK8 solution (10%) was added at 0 h and 24 h after the normal culture medium was used, and the culture was continued in the incubator for 1 h;
(4) after 1 h, the absorbance at 450 nm of each well was measured using a microplate reader. The relative survival rate of the cells was calculated according to the measured OD value. Cell viability=(material added group−blank group)/(control group−blank group)×100%. FIGS. 3A-3B show biological safety of AP1 at 24 h and 48 h after incubation in B16F10 cells for 24 h. FIG. 3A shows the inhibition of cell growth at 24 h after incubation of AP1 in B16F10 cells for 24 h, and FIG. 3B shows the inhibition rate of cell growth at 48 h after incubation of AP1 in B16F10 cells for 24 h.

Safety of radiolabeled ⁶⁸Ga-AP1 imaging probe
(1) The B16F10 cells of mice in the logarithmic growth phase were taken and digested by using 0.25% pancreatin, and the cell concentration was adjusted to 6×10⁴ cells/mL; the cells were plated in a 96-well plate at a volume of 100 μL per well, the cell concentration was 6×10³ cells per well, and the cells were incubated overnight in an incubator at 37° C./5% CO₂;
(2) the medium was replaced for each group of cells, 100 μL of a culture solution with a specified concentration was added, and the radioactive doses were taken as 0, 1, 2.5, 10, 25, 50, 100, and 200 μCi/mL; after 12 h, the normal culture medium was used and 10 μL of CCK8 solution (10%) was added, and the culture was continued in an incubator for 1 h;
(3) after 1 h, the absorbance at 450 nm of each well was measured using a microplate reader. The relative survival rate of the cells was calculated according to the measured OD value. Cell viability=(material added group−blank group)/(control group−blank group)×100%. FIG. 4 shows biological safety of ⁶⁸Ga-AP1 at different doses.

### EXPERIMENTAL EXAMPLE 2

### Cell Binding and Inhibition Assay

### Binding and Inhibition Assay of B16F10 Cells to ⁶⁸Ga-AP1

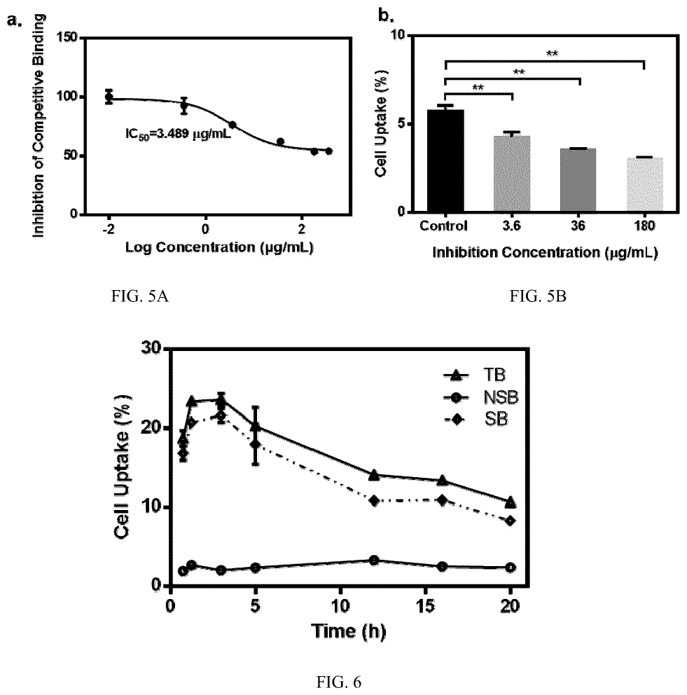

(1) When the cells grew to more than 90%, the cells were digested with 0.25% pancreatin, the cell concentration was adjusted to 1×10⁵ cells/mL, the cells were plated in a 24-well plate, and 0.5 mL of culture medium was added to each well. The cells were incubated overnight in an incubator at 37° C./5% CO₂.
(2) On the next day, after the cells were completely adhered to the wall, the culture medium added with 0.11 MBq/mL ⁶⁸Ga-AP1 and different concentrations of AP1 (0, 0.36, 3.6, 36, 180, and 360 μg/mL) was used, and 3 replicate wells were set up in each group.
(3) The culture medium added with the radiolabeled drug was incubated with B16F10 cells for 1 h.
(4) After the supernatant was removed and the cells were washed twice with PBS, the cells at the bottom of the well were lysed with 0.5 mL of 0.2 M NaOH and washed with PBS, all cells were collected in a radioimmunotube. 0.5 mL of 0.11 MBq/mL ⁶⁸Ga-AP1 was the source count T.
(5) The radioactivity of the liquid (⁶⁸Ga-AP1) detected and collected by the radioimmunotube of each concentration of AP1 was denoted as count B.
(6) The cellular uptake was B/T×100%, and the half inhibitory concentrations were fitted based on different concentrations of AP1. FIGS. 5A-5B show a competitive binding and inhibition experiment between ⁶⁸GaAP1 and AP1.

### In Vitro Targeting Verification of ¹⁷⁷Lu-AP1

(1) When the B16F10 cells grew to more than 90%, the cells were digested with 0.25% pancreatin, the cell concentration was adjusted to 1×10⁵ cells/mL, the cells were plated in a 24-well plate, and 0.5 mL of culture medium was added to each well. The cells were incubated overnight in an incubator at 37° C./5% CO₂.
(2) On the next day, after the cells were completely adhered to the wall, the culture medium added with 0.11 MBq/mL ¹⁷⁷Lu-AP1 and excessive AP1 (100 μg/mL) was used, and 2 replicate wells were set up in each group.
(3) The medium added with the radiolabeled drug was allowed to act on B16F10 cells for different periods of time, and an inhibition group was set for each time point.
(4) At different time points, the supernatants were removed, the cells were washed twice with PBS, the cells at the bottom of the wells were lysed with 0.5 mL of 0.2 M NaOH, collected and washed with PBS, and all cells were collected in a radioimmunotube. 0.5 mL of 0.11 MBq/mL of ¹⁷⁷Lu-AP1 was the source count T.
(4) The total radioactive binding of the liquid (count B) detected and collected by the radioimmunotube of each concentration of AP1 was denoted as TB, the non-specific binding of the uptake rate inhibited by the addition of excess AP1 was denoted as NSB, and the specific binding was denoted as SB=TB−NSB. FIG. 6 shows a specific uptake and inhibition of ¹⁷⁷Lu-AP1 in B16F10 cells.

### EXPERIMENTAL EXAMPLE 3

### Experiment of Growth Inhibitory Effect of ¹⁷⁷Lu-AP1 on B16F10 Cells

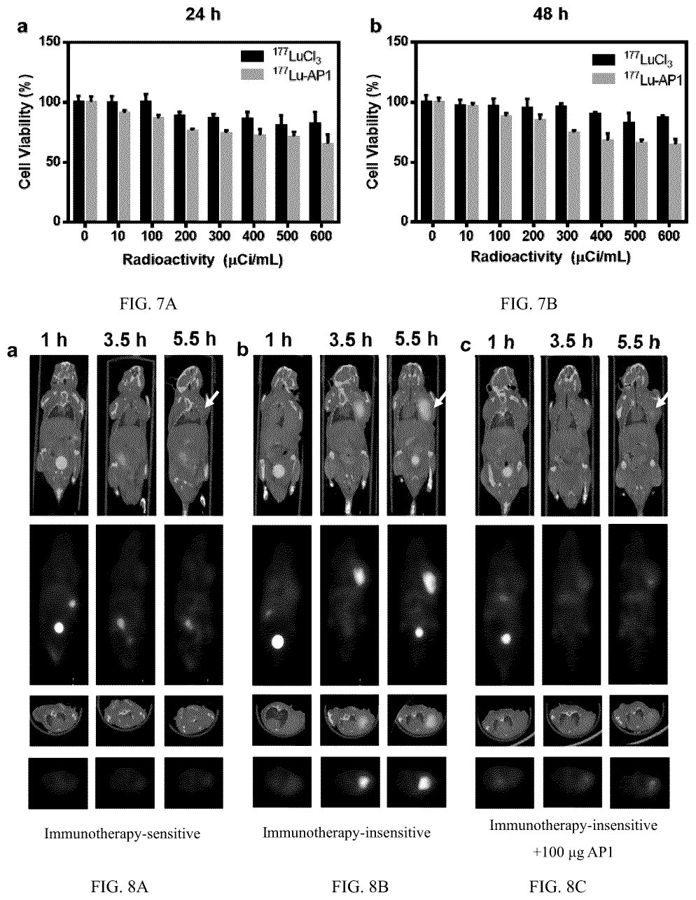

(1) The B16F10 cells of mice in the logarithmic growth phase were taken and digested by using pancreatin, and the cell concentration was adjusted to 4×10⁴ cells/mL; the cells were plated in a 96-well plate at a volume of 100 μL per well, the cell concentration was 4×10³ cells per well, and the cells were incubated overnight in an incubator at 37° C./5% CO₂;
(2) the medium was replaced for each group of cells, 100 μL of culture solution with a specified dosage was added, and ¹⁷⁷LuCl₃ and ¹⁷⁷Lu-AP1 with the same dosage concentration were added into two 96-well plates, wherein dosage concentrations were 0, 10, 100, 200, 300, 400, 500 and 600 μCi/mL; after 24 h, the normal culture medium was used to continue culturing;
(3) a culture medium with 10 μL of CCK8 solution (10%) was added at 24 h and 48 h after the normal culture medium was used, and the culture was continued in the incubator for 1 h;
(4) after 1 h, the absorbance at 450 nm of each well was measured using a microplate reader. The relative survival rate of the cells was calculated according to the measured OD value. Cell viability=(material added group−blank group)/(control group−blank group)×100%. FIGS. 7A-7B show inhibitory effect of ¹⁷⁷Lu-AP1 on B16F10 cell growth, where FIG. 7A shows cell growth inhibition rate at 24 h after incubation for 24 h, and FIG. 7B shows cell growth inhibition rate at 48 h after incubation for 24 h.

### EXPERIMENTAL EXAMPLE 4

### Verification of In Vivo Targeting and Specificity of ⁶⁸Ga-AP1 Imaging Probe

(1) The B16F10 cells in the logarithmic growth phase were trypsinized and resuspended to a concentration of 1×10⁶ cells/mL, and the amount of cells injected per mouse was 1×10⁵ in 6-week-old C57BL/6 tumor-bearing left forelimbs.
(2) On the day 10 after tumor bearing, 30-100 μCi of ⁶⁸Ga-AP1 was injected into tail veins of different mice, and Micro-PET/CT static development was performed for 10 min 1.5, 3.5 and 5.5 h after the administration (after the isoflurane-oxygen mixed gas with a volume fraction of 3% was used for preanesthesization, the mice were placed in a PET/CT scanning bed, and then the isoflurane-oxygen mixed gas with a volume fraction of 1.5% was used for maintaining the anesthesia).
(3) Highly expressed tumors were imaged and injected with the same dose of ⁶⁸Ga-AP1 and 100 μg AP1 the next day for in vivo uptake inhibition experiments, and Micro-PET/CT static imaging was performed for 10 min at 1, 3.5, and 5.5 h after intravenous injection, where the white arrows were tumors. FIGS. 8A-8C show in vivo targeting and specificity of a ⁶⁸Ga-AP1 imaging probe, where FIG. 8A shows PET/CT imaging of an individual that is sensitive to immunotherapy; FIG. 8B shows PET/CT imaging of an individual that is not insensitive to immunotherapy, and FIG. 8C shows PET/CT imaging of a high-uptake tumor that is insensitive to immunotherapy after addition of AP1 inhibition.

### EXPERIMENTAL EXAMPLE 5

### In Vivo Biodistribution and Hemodynamic Characteristics of ⁶⁸Ga-AP1 Imaging Probe

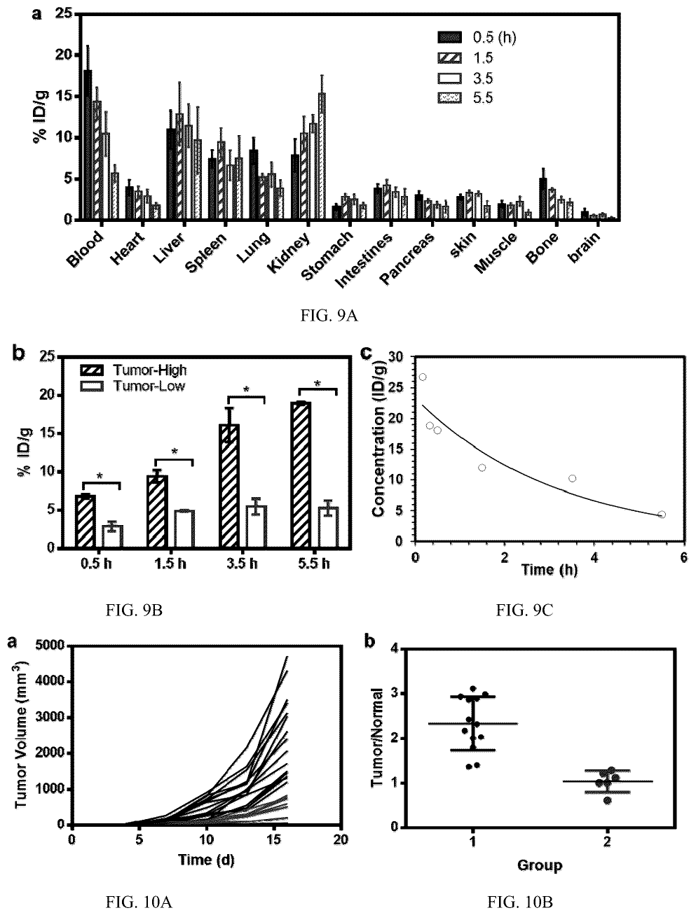

(1) The ⁶⁸Ga-AP1 imaging probe was used for screening a high expression group and a low expression group of tumor ASF1a, and 20 μCi of ⁶⁸Ga-AP1 was injected into the tail vein of each mouse. After 0.5, 1.5, 3.5 and 5.5 h after injection, each important organ was dissected and separated. Each organ weight was measured and calculated as % ID/g=organ radioactivity count/(organ weight×source count)×100% for 3 mice per time point.
(2) Mice were sacrificed at 10 min, 20 min, 30 min, 1.5 h, 3.5 h, 5.5 h respectively after tail vein injection of ⁶⁸Ga-AP1, the radioactivity of the organs in the blood of the mice was measured, n=3 was measured at each time point, and the mean value of each time point was taken, and an intravascular two-compartment model was selected to fit hemodynamic parameters using PKSover software. FIGS. 9A-9C show in vivo biodistribution and hemodynamic characteristics of a ⁶⁸Ga-AP1 imaging probe, where FIG. 9A shows biodistribution of 68Ga-AP1 in major organs in a body, FIG. 9B shows distributions of ⁶⁸Ga-AP1 in ASF1a high-expression and low-expression tumors, respectively, and FIG. 9C shows pharmacokinetics of ⁶⁸Ga-AP1 in vivo.

### EXPERIMENTAL EXAMPLE 6

### Evaluation of Correlation Between Different Expressions of ASF1a and the Efficacy of Immunotherapy in a Melanoma B16F10 Model

(1) The B16F10 tumor-bearing mouse model was established, and each mouse was born with 1×10⁵ cells in the left forelimb. From day 4, 0.1 mg of BMS-1 was injected intraperitoneally every 3 days, and the tumor volume was measured; from day 7, PET imaging was performed every 3 days, and the ratio of tumor uptake/contralateral muscle uptake was recorded.
(2) When the tumor grew to day 16, the tumor volume was less than 1000 mm³, which was regarded as the immunotherapy-effective group (immunotherapy-sensitive group), and the correlation of ⁶⁸Ga-AP1 PET imaging uptake in the immunotherapy-sensitive group and the immunotherapy-insensitive group was compared. FIGS. 10A-10B show the correlation between different expressions of ASF1a and the efficacy of immunotherapy in a melanoma B16F10 model, where FIG. 10A shows a tumor volume growth curve of mice after treatment with the immunosuppressant BMS-1, purple represents an effective immunosuppressive treatment group, and black represents an ineffective immunosuppressive treatment group; FIG. 10B shows ratios of the tumors to contralateral muscle uptake in different response groups of immunotherapy.

### EXPERIMENTAL EXAMPLE 7

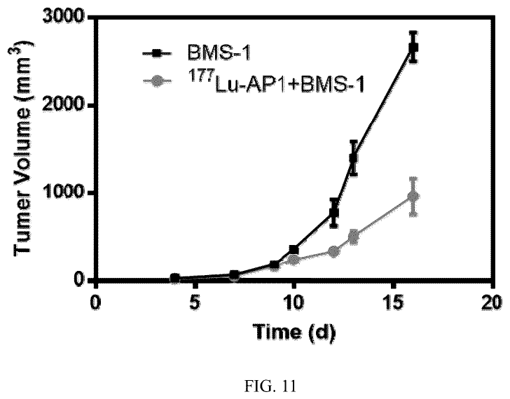

Individuals with high expression of ASF1a predicted to be insensitive to immunotherapy were screened by early ⁶⁸Ga-AP1 PET imaging, and ¹⁷⁷Lu-AP1 targeted radionuclide therapy was performed and the efficacy was evaluated
(1) A B16F10 tumor mouse model was established, the 68Ga-AP1 PET imaging was performed on days 7-8 after treatment, individuals with high expression of ASF1a were screened, one group was administrated with PDL1 inhibitor BMS-1, and the other group was administrated with BMS-1 combined with ¹⁷⁷Lu-AP1. The tumor growth was measured and recorded every 2-3 days. FIG. 11 shows tumor volume growth curves of immunotherapy-insensitive individuals treated with BMS-1 alone and BMS-1 combined with ¹⁷⁷Lu-AP1.

The present invention establishes a method for labeling ASF1a inhibitory peptide (AP1) with ⁶⁸Ga/¹⁷⁷Lu by using ⁶⁸GaCl₃ produced by a ⁶⁸Ge—⁶⁸Ga generator or purchased ¹⁷⁷LuCl₃ and designed ASF1a inhibitory peptide, evaluates the pharmacological characteristics and the biological characteristics of the ASF1a inhibitory peptide in a B16F10 tumor model mouse, is further used in the ASF1a targeted imaging study, and analyzes the correlation of the imaging result with immunotherapy. The efficacy of radionuclide-targeted therapy on screened ASF1a individuals is evaluated. The preclinical study shows that the labeling rate of ⁶⁸Ga-AP1 is 81.98±7.55%, the labeling rate of ¹⁷⁷Lu-AP1 is 78.34±13.59%, the radiochemical purity of the product measured by an HPLC method is greater than 95%, and the product has good stability within 24 h.

The above descriptions are only preferred embodiments of the present invention. It should be noted that those of ordinary skill in the art can also make several improvements and modifications without departing from the principle of the present invention, and such improvements and modifications shall fall within the protection scope of the present invention.

What is claimed is:

1. A nuclide-labeled inhibitory peptide, prepared by labeling an ASF1a peptide with ⁶⁸Ga or ¹⁷⁷Lu by 1,4,7,10-tetraazacyclododecane-1,4,7,10-tetraacetic acid (DOTA), wherein an amino acid sequence of the ASF1a peptide is YGRKKRRQRRRCASTEEKWARLARRIAGAGGVTLDGFGGCA, as shown in SEQ ID NO: 1.

2. A preparation method for the nuclide-labeled inhibitory peptide according to claim 1, comprising the following steps:
(1) mixing a DOTA-coupled ASF1a peptide with a nuclide solution to obtain a mixed solution, mixing the mixed solution with sodium acetate to obtain a resulting solution, adjusting a pH of the resulting solution, and placing the resulting solution in a water bath to obtain a reaction solution;
(2) enabling the reaction solution obtained in step (1) to pass through a chromatography column, and collecting a product.

3. The preparation method for the nuclide-labeled inhibitory peptide according to claim 2, wherein the nuclide solution in step (1) is a ⁶⁸GaCl₃ solution or a ¹⁷⁷LuCl₃/HCl solution, and a radiation amount of the ⁶⁸GaCl₃ solution or the ¹⁷⁷LuCl₃/HCl solution is independently 111-185 MBq.

4. The preparation method for the nuclide-labeled inhibitory peptide according to claim 3, wherein a preparation method for the ⁶⁸GaCl₃ solution comprises: rinsing a ⁶⁸Ge—⁶⁸Ga generator by hydrochloric acid, and collecting an intermediate product ⁶⁸GaCl₃; wherein an amount of the hydrochloric acid is 4 mL, and the intermediate product is ⁶⁸GaCl₃, and the ⁶⁸GaCl₃ is 2^{nd} to 3^{rd} mL flowing out of the ⁶⁸Ge—⁶⁸Ga generator.

5. The preparation method for the nuclide-labeled inhibitory peptide according to claim 4, wherein the hydrochloric acid has a concentration of 0.04-0.06 M, and the ⁶⁸Ge—⁶⁸Ga generator has a flow rate of 0.8-1.2 mL/min.

6. The preparation method for the nuclide-labeled inhibitory peptide according to claim 2, wherein the DOTA-coupled ASF1a peptide in step (1) has a concentration of 0.8-1.2 mg/mL, and a volume ratio of the DOTA-coupled ASF1a peptide to the nuclide solution is (0.01-0.03):(1.00-3.00).

7. The preparation method for the nuclide-labeled inhibitory peptide according to claim 2, wherein a volume ratio of the DOTA-coupled ASF1a peptide to the sodium acetate in step (1) is (15-25):(250-350), the sodium acetate has a concentration of 0.23-0.27 M, and the pH is adjusted to 3.8-4.2.

8. The preparation method for the nuclide-labeled inhibitory peptide according to claim 2, wherein the water bath in step (1) has a temperature of 93-97° C., and the chromatography column in step (2) is a C18 small column.

9. The preparation method for the nuclide-labeled inhibitory peptide according to claim 3, wherein when the nuclide solution is the ⁶⁸GaCl₃ solution, the water bath is performed for 8-12 min, and when the nuclide solution is the ¹⁷⁷LuCl₃/HCl solution, the water bath is performed for 25-35 min.

10. A method for preparing an anti-tumor drug, comprising: using the nuclide-labeled inhibitory peptide according to claim 1, wherein the nuclide-labeled inhibitory peptide is a nuclide ¹⁷⁷Lu-labeled inhibitory peptide, and a tumor is one of melanoma, lung cancer, lung metastatic cancer, and breast cancer.

11. A method for preparing a PET/CT imaging agent, comprising: using the nuclide-labeled inhibitory peptide according to claim 1, wherein the nuclide-labeled inhibitory peptide is a nuclide ⁶⁸Ga-labeled inhibitory peptide.

12. A method for preparing an anti-tumor drug, comprising: using a nuclide-labeled inhibitory peptide prepared by the preparation method for the nuclide-labeled inhibitory peptide according to claim 2, wherein the nuclide-labeled inhibitory peptide is a nuclide ¹⁷⁷Lu-labeled inhibitory peptide, and a tumor is one of melanoma, lung cancer, lung metastatic cancer, and breast cancer.

13. The method for preparing the anti-tumor drug according to claim 12, wherein in the preparation method for the nuclide-labeled inhibitory peptide, the nuclide solution in step (1) is a ⁶⁸GaCl₃ solution or a ¹⁷⁷LuCl₃/HCl solution, and a radiation amount of the ⁶⁸GaCl₃ solution or the ¹⁷⁷LuCl₃/HCl solution is independently 111-185 MBq.

14. The method for preparing the anti-tumor drug according to claim 13, wherein a preparation method for the ⁶⁸GaCl₃ solution comprises: rinsing a ⁶⁸Ge—⁶⁸Ga generator by hydrochloric acid, and collecting an intermediate product ⁶⁸GaCl₃; wherein an amount of the hydrochloric acid is 4 mL, and the intermediate product is ⁶⁸GaCl₃, and the ⁶⁸GaCl₃ is 2^{nd} to 3^{rd} mL flowing out of the ⁶⁸Ge—⁶⁸Ga generator.

15. The method for preparing the anti-tumor drug according to claim 14, wherein the hydrochloric acid has a concentration of 0.04-0.06 M, and the ⁶⁸Ge—⁶⁸Ga generator has a flow rate of 0.8-1.2 mL/min.

16. The method for preparing the anti-tumor drug according to claim 12, wherein in the preparation method for the nuclide-labeled inhibitory peptide, the DOTA-coupled ASF1a peptide in step (1) has a concentration of 0.8-1.2 mg/mL, and a volume ratio of the DOTA-coupled ASF1a peptide to the nuclide solution is (0.01-0.03):(1.00-3.00).

17. The method for preparing the anti-tumor drug according to claim 12, wherein in the preparation method for the nuclide-labeled inhibitory peptide, a volume ratio of the DOTA-coupled ASF1a peptide to the sodium acetate in step (1) is (15-25):(250-350), the sodium acetate has a concentration of 0.23-0.27 M, and the pH is adjusted to 3.8-4.2.

18. The method for preparing the anti-tumor drug according to claim 12, wherein in the preparation method for the nuclide-labeled inhibitory peptide, the water bath in step (1) has a temperature of 93-97° C., and the chromatography column in step (2) is a C18 small column.

19. The method for preparing the anti-tumor drug according to claim 13, wherein when the nuclide solution is the ⁶⁸GaCl₃ solution, the water bath is performed for 8-12 min, and when the nuclide solution is the ¹⁷⁷LuCl₃/HCl solution, the water bath is performed for 25-35 min.

20. A method for preparing a PET/CT imaging agent, comprising: using a nuclide-labeled inhibitory peptide prepared by the preparation method for the nuclide-labeled inhibitory peptide according to claim 2, wherein the nuclide-labeled inhibitory peptide is a nuclide ⁶⁸Ga-labeled inhibitory peptide.


SEQUENCE LISTING (1 sequence)

SEQ ID NO: 1 - protein, 41 aa, synthetic construct
  REGION 1..41: note = The sequence is synthetized.
YGRKKRRQRRRCASTEEKWARLARRIAGAGGVTLDGFGGCA